(19) United States
(12) Patent Application Publication
(10) Pub. No.: US 2020/0390321 A1
(43) Pub. Date: Dec. 17, 2020

(54) Hysteroscope Cross-Sections For Diagnostic And Therapeutic Procedures And Method Thereof

(71) Applicant: Caldera Medical, Inc., Agoura Hills, CA (US)

(72) Inventors: Jose Luis Charvet, Agoura Hills, CA (US); Vishtasb Farkhondeh, Agoura Hills, CA (US)

(73) Assignee: Caldera Medical, Inc., Agoura Hills, CA (US)

(21) Appl. No.: 16/900,496

(22) Filed: Jun. 12, 2020

Related U.S. Application Data
(60) Provisional application No. 62/861,184, filed on Jun. 13, 2019.

(51) Int. Cl. A61B 1/018; A61B 17/42; A61B 1/07; A61B 1/12; A61B 1/002

(52) U.S. Cl. CPC A61B 1/018; A61B 17/42; A61B 1/07; A61B 2017/0034; A61B 1/002; A61B 2217/005; A61B 2017/4216; A61B 1/12

(57) ABSTRACT

The present disclosure relates to medical devices. More particularly, this disclosure describes a hysteroscope having a multi-lumened elongated distal outer tube extending from a proximate body to a distal portion. The hysteroscope may allocate space for an optical, luminescence, irrigation and working lumens that substantially extend through the outer tube. These lumens, or channels, as provided in this disclosure will be defined in different cross-sections of the distal outer tube maximizing the area therein. In an illustrative example, an ellipse-shaped lumen surrounded by a circular outer tube defines one such cross-section and may include the optical and working lumens with light fibers positioned between the lumens all within the ellipse-shaped lumen. Exterior to the ellipse-shaped lumen may be a first and second irrigation lumen.

### RELATED DISCLOSURE

This disclosure claims priority to U.S. Provisional Application Ser. No. 62/861,184 filed Jun. 13, 2019, titled Hysteroscope Cross-Sections for Therapeutic Procedures, which is hereby incorporated by reference in its entirety.

### TECHNICAL FIELD

The present disclosure relates to the field of uterine tissue diagnosis and therapeutic treatments and, more particularly, to a hysteroscopy system having various cross-sectional configurations of multiple lumens that are driven by geometric constraints to separate optical, luminescence, irrigation and/or working channels.

### BACKGROUND

During the last five decades, medical technology development has increased. For example, rigid endoscopes for diagnostic procedures, such as hysteroscopes, have evolved from a simple slender instrument to having an optical system with a fiber optic illumination system. The use of hysteroscopes and other manual surgical instruments allowed therapeutic procedures in the past. However, it is not until the last decade when hysteroscopes have been used in conjunction with powered tissue removal devices to surgically remove pathologies inside a uterus.

Hysteroscopes typically include a sheath, scope or barrel and various lumens defining channels for fluid control. A working lumen for insertion of therapeutic instruments, such as tissue removal devices may also be incorporated into the hysteroscopes. Powered surgical tools, such as a morcellator, may be inserted into the working lumen. At a minimum, the hysteroscope may allocate space for an optical, luminescence, irrigation and working lumens.

Complications arise from the hysteroscope's size. The size of the hysteroscope that enters the uterus may drive the design of these lumens and the scope or barrel, which may be interpreted by their cross-sections. These design elements may affect functions of the hysteroscope. Typically, the smaller the cross-section of the hysteroscope, the more comfortable the patient. Oppositely, a larger optical lumen may produce better image quality and a bigger luminescence may provide better quality lighting. In addition, a wider irrigation lumen may enable better fluid balance inside the uterus and a larger working channel may allow for higher capacity surgical tools which may result in a faster procedure.

The present disclosure provides for hysteroscope cross-sections for diagnostic and therapeutic procedures and methods thereof that addresses the above identified concerns. Other benefits and advantages will become clear from the disclosure provided herein and those advantages provided are for illustration. The statements in this section merely provide the background related to the present disclosure and does not constitute prior art.

### SUMMARY

This summary is provided to introduce a selection of concepts in a simplified form that are further described below in the DESCRIPTION OF THE DISLCOSURE. This summary is not intended to identify key features of the claimed subject matter, nor is it intended to be used as an aid in determining the scope of the claimed subject matter.

According to one aspect of the present disclosure, a hysteroscope is provided. The hysteroscope may include a proximal body from which a multi-lumened elongated outer tube extends, an ellipse-shaped lumen positioned inside the outer tube, an optical lumen disposed within the ellipse-shaped lumen, a working lumen disposed within the ellipse-shaped lumen, light fibers positioned within the ellipse-shaped lumen, a first irrigation lumen positioned outside the ellipse-shaped lumen and within the outer tube, and a second irrigation lumen positioned outside the ellipse-shaped lumen and within the outer tube.

According to another aspect of the present disclosure, a hysteroscopy system is provided. The hysteroscopy system may include a body and a multi-lumened elongated outer tube extending from the body. The outer tube may include an optical lumen extending through the outer tube, a working lumen extending through the outer tube, at least one luminescence extending through the outer tube, a first irrigation lumen positioned within the outer tube, and a second irrigation lumen positioned the outer tube.

According to yet another aspect of the present disclosure, a method of accessing an internal site in a patient's uterus is provided. The method may include positioning the following lumens of a hysteroscope into a patient's uterus: an ellipse-shaped lumen having an optical lumen and working lumen positioned therein; and at least one irrigation lumen positioned outside the ellipse-shaped lumen. The method may also include introducing inflow fluid into the patient's uterus through the at least one irrigation lumen and thereby distending the patient's uterus and suctioning at least a portion of the inflow fluid out of the uterus through the working lumen.

### BRIEF DESCRIPTION OF THE DRAWINGS

The novel features believed to be characteristic of the disclosure are set forth in the appended claims. In the descriptions that follow, like parts are marked throughout the specification and drawings with the same numerals, respectively. The drawing FIGURES are not necessarily drawn to scale and certain FIGURES may be shown in exaggerated or generalized form in the interest of clarity and conciseness. The disclosure itself, however, as well as a preferred mode of use, further objectives and advantages thereof, will be best understood by reference to the following detailed description of illustrative embodiments when read in conjunction with the accompanying drawings, wherein:

FIG. 1 is a top perspective view of an illustrative hysteroscope according to one embodiment of the present disclosure;

FIG. 2 is a cross-sectional view of a patient's uterus with a portion of the illustrative hysteroscope inserted therein according to one embodiment of the present disclosure;

FIG. 3A is a top perspective view of a distal end of the illustrative hysteroscope having an ellipse-shaped lumen according to one embodiment of the present disclosure;

FIG. 3B is a side view into a barrel of the illustrative hysteroscope without a surgical tool according to one embodiment of the present disclosure;

FIG. 3C is a side view into the barrel of the illustrative hysteroscope with the surgical tool inserted according to one embodiment of the present disclosure;

FIG. 3D is a cross-sectional side view of the barrel with the surgical tool inserted into the illustrative hysteroscope according to one embodiment of the present disclosure;

FIG. 4 is a top perspective view of a cross-section of the barrel of the illustrative hysteroscope with the surgical tool inserted showing a regulation of inflow and outflow of fluid according to one embodiment of the present disclosure;

FIG. 5 is a top perspective view of a cross-section of a barrel of another illustrative hysteroscope having an amorphous-shaped lumen according to one embodiment of the present disclosure;

FIG. 6 is a top perspective view of a cross-section of a barrel of another illustrative hysteroscope having ellipse-shaped irrigation lumens according to one embodiment of the present disclosure;

FIG. 7 is a top perspective view of a cross-section of another illustrative hysteroscope having an oblong-shaped barrel according to one embodiment of the present disclosure;

FIG. 8 is a top perspective view of a cross-section of another illustrative hysteroscope having a pear-shaped barrel according to one embodiment of the present disclosure; and

FIG. 9 is an exemplary flow chart showing illustrative processes for accessing an internal site in a patient's uterus according to one embodiment of the present disclosure.

### DESCRIPTION OF THE DISCLOSURE

The description set forth below in connection with the appended drawings is intended as a description of exemplary embodiments of the disclosure and is not intended to represent the only forms in which the present disclosure may be constructed and/or utilized. The description sets forth the functions and the sequence of blocks for constructing and operating the disclosure in connection with the illustrated embodiments. It is to be understood, however, that the same or equivalent functions and sequences may be accomplished by different embodiments that are also intended to be encompassed within the spirit and scope of this disclosure.

Generally described, the present disclosure relates to medical devices. More particularly, this disclosure describes a hysteroscope having a multi-lumened elongated distal outer tube extending from a proximate body to a distal portion. The hysteroscope may allocate space for an optical, luminescence, irrigation and working lumens that substantially extend through the outer tube. These lumens, or channels, as provided in this disclosure will be defined in different cross-sections of the distal outer tube that maximize the area therein. In an illustrative example, an ellipse-shaped lumen surrounded by a circular outer tube defines one such cross-section and may include the optical and working lumens with light fibers positioned between the lumens all within the ellipse-shaped lumen. Exterior to the ellipse-shaped lumen may be a first and second irrigation lumen.

Numerous other modifications or configurations to the cross-sections of the distal outer tube will become apparent from the description provided below. For example, the outer tube may be oblong-shaped or pear-shaped. The optical, luminescence, irrigation and working lumens may be placed in different formations based on geometrical constraints. Advantageously, the different cross-sections may keep the same circular cross-sectional structures for the optical and working lumens which may have standardized tool sets for the hysteroscope. It may also lead to a more simplified or streamlined manufacturing processes. Other advantages will become apparent from the description provided below.

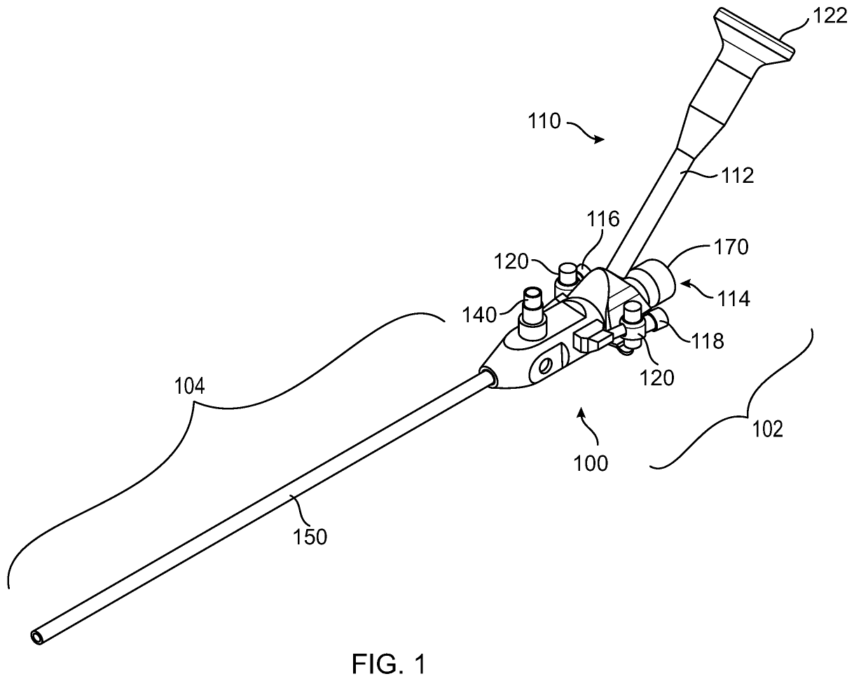

Turning to FIG. 1, a top perspective view of an illustrative hysteroscope 100 according to one embodiment of the present disclosure is provided. The hysteroscope 100 may include, but is not limited to, an operation section or body 102 at a proximal portion and an elongated tube, or sheath, as an insertion section 104 at the distal portion. The hysteroscope 100 may be a five-lumened apparatus having an optical system 110 to enable viewing of a pathology, a connector for a light source to illuminate the area of interest inside the uterus 200, a working channel for accepting a tissue resector or other instrument and for facilitating fluid outflow, for example, via a vacuum, and two independent irrigation or inflow channels associated with valves 120, that work in conjunction with the central channel out flow element to distend the uterus 200 during diagnostic (pathology identification) and therapeutic (pathology removal) procedures.

In certain embodiments, the hysteroscope 100 may function both as a diagnostic (pathology identification) and therapeutic (pathology removal) tool. In one embodiment for the diagnostic procedure, the hysteroscope 100 may be configured to seal the working channel from fluid transport and employ one irrigation channel for fluid inflow and another irrigation channel for fluid outflow, e.g. via a vacuum source. This configuration may negate the need for employing a modular outflow channel. If a tissue pathology is identified during the diagnostic procedure, the configuration of the hysteroscope 100 may receive a resector which may be inserted into the working channel for removal of the relevant tissue. When switching to the therapeutic portion, both inflow and outflow tubes may be removed from the irrigation channels and may be replaced with a custom Y-tubing providing fluid inflow.

The operation section or the body 102 at the proximal end of the hysteroscope 100 may include an optical system 110 employing an optical or first lumen 112. As shown, the proximal end 122 of the first lumen 112 may protrude upwardly from the body 102 towards a viewer which is provided with an optical output or eyepiece at the terminal end for user observation. Alternatively, the proximal end 122 of the first lumen 112 may employ an optical cable coupling element for connection and image viewing on a remote screen. A distal portion of the first lumen 112 may be located inside the insertion section 104. The optical system 110 may enable optimal viewing of the pathology by, for example, housing a train of rod lenses and spacers inside the first lumen 112. The optical system 110 may also be, for example, a bead-lens system or graded index system. A distal end of the first lumen 112 may be sealed to prevent entry of fluid into the lumen 112.

A second lumen 114 may be located at the proximal portion of the body 102 underneath the first lumen 112. The second lumen 114 may extend into insertion section 104 at its distal portion. The second lumen 114 may function as a working channel, for example, for receiving an instrument such as a single-use tissue removal device (TRD) or tissue resector. The second lumen 114 may have an opening at a distal and proximal end. A seal 170 at a proximal portion of the second lumen 114 may allow a fluid sealing and/or a friction fit with the inserted instrument, for example, a tissue resector and/or a modular outflow channel. The second lumen 114 provides for an outflow channel.

The proximal portion of the body 102 of the hysteroscope 100 may further include a third lumen 116 and a fourth lumen 118 for irrigation, wherein both irrigation lumens may extend through and to a distal end of the insertion section 104 of the hysteroscope 100. The third lumen 116 and fourth lumen 118 may be located laterally and symmetrically between the first lumen 112 and the second lumen 114. The third lumen 116 and fourth lumen 118 may be independent irrigation lumens or channels having openings at the distal end of the body 102 and valves 120 at their proximal portions to control the flow of fluid therethrough in order to keep the uterus distended and pressure maintained with a fluid medium during diagnostic and therapeutic procedures.

The insertion section 104 of the hysteroscope 100 may include a single tubing or barrel 150 enclosing the distal portions and ends of the first lumen 112 of the optical system, the second lumen 114 for the working channel, and the third lumen 116 and fourth lumen 118 of the irrigation channels. At least one space or cavity may be created by the inner wall of the barrel 150 and the outer walls of the four lumens. The cavity may be completely or partially occupied by a light transmission element or elements, for example, by fiber optic cables or bundles connected to a light post 140 proximally positioned on the body 102 of the hysteroscope 100. At the distal end of the barrel 150, the cavity may be sealed from fluid entry, for example, by a clear or transparent adhesive, so that light may be transmitted from the light post 140 out the distal end of the barrel 150 and into the uterus. In an alternative embodiment, and as will be seen below, the cavity having the light transmission element or elements may themselves be provided in at least one lumen.

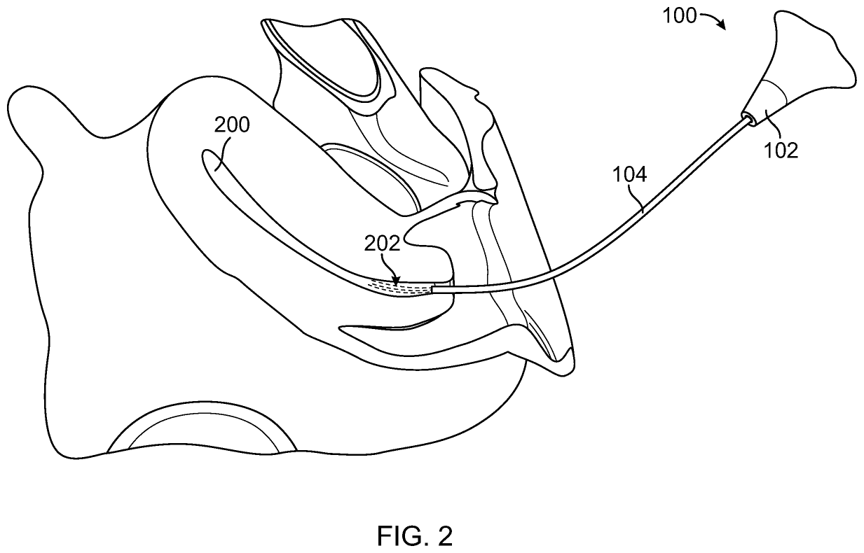

FIG. 2 is cross-sectional view of a patient's uterus 200 with a portion of the illustrative hysteroscope 100 inserted therein according to one embodiment of the present disclosure. The insertion section 104 through the multiple lumens may deliver continuous fluid irrigation to the uterus 200 both to distend it for visibility and safety as well as to remove and cut pathologies.

In the case of gynecological procedures, the second lumen 114 within the insertion section 104 may provide outflow of fluid while allowing for various surgical instruments to be inserted and retracted through the working channel. This configuration may allow the insertion section 104 to have a small cross-sectional profile and/or a slim profile, which may minimize discomfort, trauma, and/or injury to the patient during a gynecological procedure. If the insertion section 104 is entering the cervix 202, then an insertion section 104 having a smaller cross-sectional profile may cause less pain to the patient as the insertion section 104 is inserted into the cervix 202. Typically, a smaller profile may likely require little to no cervical dilation.

As described, the insertion section 104 having the smaller cross-sectional profile may cause less pain for the patient as the insertion device section 104 is inserted into the cervix 202. One such configuration for a smaller cross-sectional profile includes an ellipse-shaped lumen fitted within the single tubing or barrel 150 of the hysteroscope 100. A number of different configurations for the lumens within the barrel 150 will now be shown of the insertion section 104. The various configurations will differ based on geometric constraints with each having their unique benefits and advantages. Certain properties, for example luminescence area and inflow-to-outflow rates, have been considered in the design of the following cross-sections.

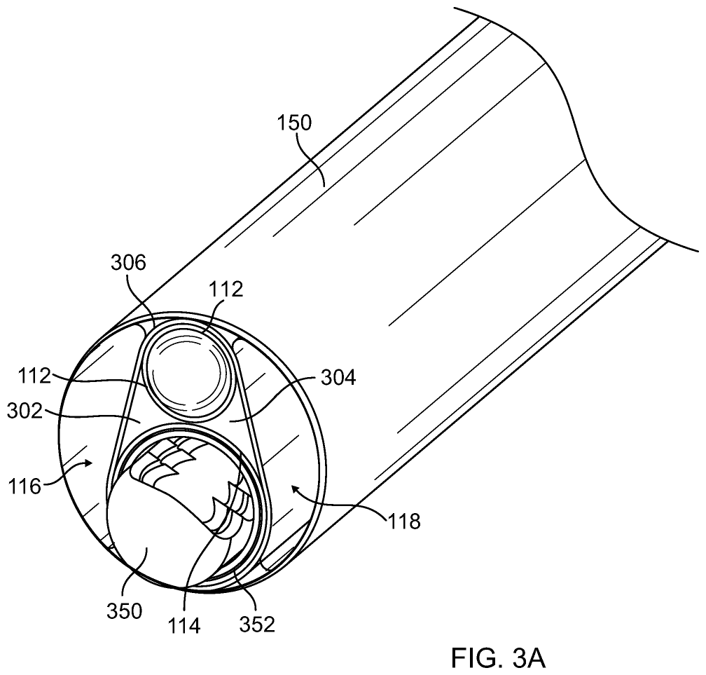

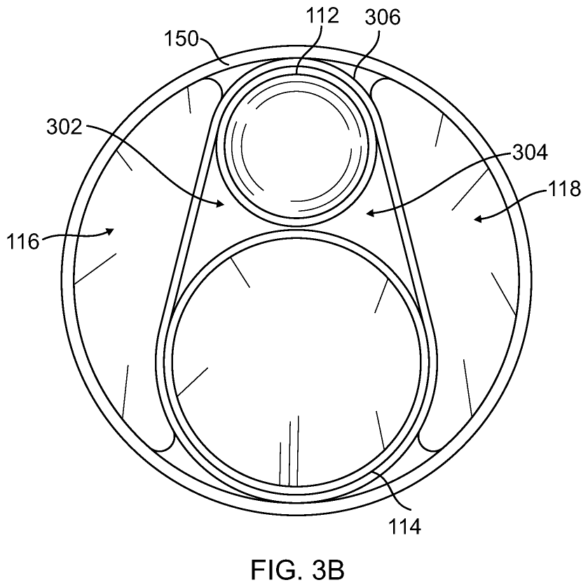

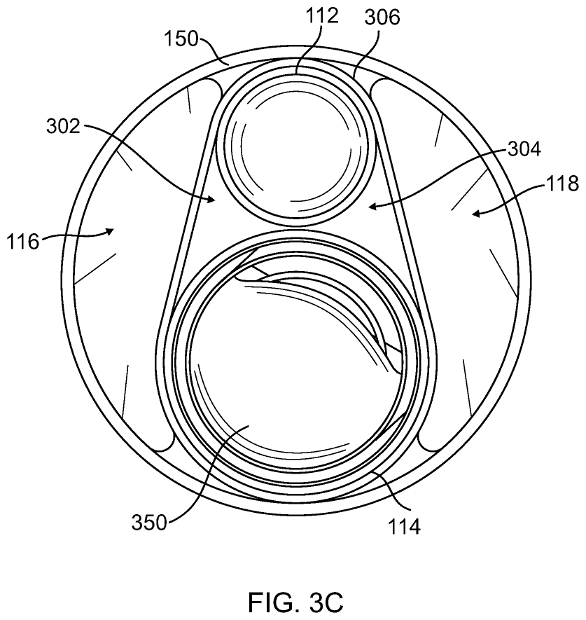

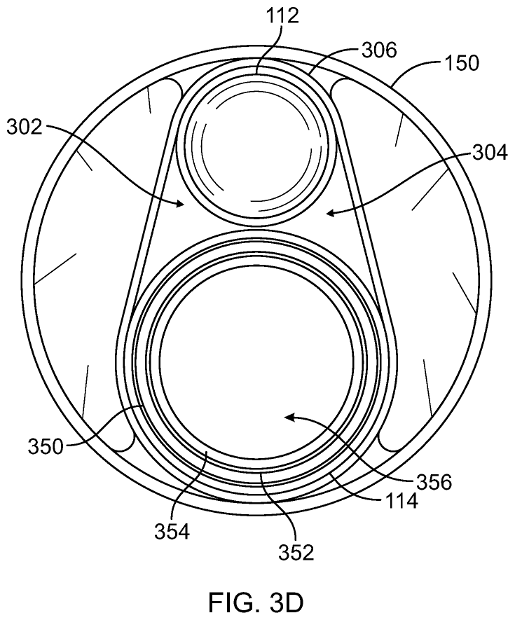

FIG. 3A is a top perspective view of the distal end of the illustrative hysteroscope 100 having an ellipse-shaped lumen 306 according to one embodiment of the present disclosure. Cross-sectional and distal end views of the single tubing or barrel 150 of the insertion section 102 of the hysteroscope 100 show the arrangement of the distal portions of the first lumen 112, second lumen 114, third lumen 116 and fourth lumen 118 inside the barrel 150.

The ellipse-shaped lumen 306 may be, for example, permanently attached to the main body or barrel 150 of the hysteroscope 100, while the outer wall of the barrel 150 may be modular in the form of a sheath. The outer sheath may be permanent and made of stainless steel, as well as the other parts found in the insertion section 104 of the hysteroscope 100. The sheath may cover the hysteroscope 100 from end-to-end isolating controls such as the optical system 110 of the hysteroscope 100 from patient contact and contamination during a procedure.

The ellipse-shaped lumen 306 of the hysteroscope 100 may be enclosed by a wall of the single tube or barrel 150. The barrel 150 may contain all the distal portions of the first lumen 112, second lumen 114, third lumen 116 and fourth lumen 118 as they substantially extend through the outer tube or barrel 150. In one example, the ellipse-shaped lumen 306 may extend an entire diameter of the interior wall of the barrel 150 coupled at a first contact point and an inferior second contact point stretched longitudinally across the barrel 150. In an alternative, the ellipse-shaped lumen 306 may be embedded into the inner diameter of the barrel 150 through known welding or fastening techniques.

As shown, the ellipse-shaped lumen 306 may be defined by the first lumen 112 of the optical system 110 and the second lumen 114 for the working channel. The first lumen 112 is located superiorly inside the ellipse-shaped lumen 306 for the optical system to enable viewing of the pathology and is fluidly sealed at the distal end. The placement of the first lumen 112 at an upper portion of the ellipse-shaped lumen 306 may lead to an easier configuration in the proximate body 102 of the hysteroscope 100, as shown in FIG. 1.

The second lumen 114 for the working channel is located, for example, inferior to the first lumen 112 with both connected to one another at a contact point stretched longitudinally across the barrel 150 and within the ellipse-shaped lumen 306. The bottom location of the second lumen 114 may be positioned as such to evacuate fluid through an outflow channel of the hysteroscope 100 that may be tied to a vacuum. Naturally falling tissue or fluid may go through the bottom-positioned second lumen 114.

The ellipse-shaped lumen 306 may be created through an exterior wall of the first lumen 112 and exterior wall of the second lumen 114 where the ellipse-shaped lumen 306 wraps or encircles both arcs of the lumens 112 and 114 with tangent lines between them. Beneficially, the ellipse-shaped lumen 306 encapsulates both the first lumen 112 and second lumen 114, which are circular in shape, and holds both to a geometric constraint defined by the diameter of the barrel 150. This constraint may set forth inflow-to-outflow rates of the hysteroscope 100 which will be shown below.

This assembly or structure may provide a stronger and more durable insertion section 104 of the hysteroscope 100. For example, an outer wall of the ellipse-shaped lumen 306 may be sized to match the inner diameter of the barrel 150 of the hysteroscope 100. A height of the inner wall of the ellipse-shaped lumen 306 may match the combined outer diameters of the first lumen 112 and second lumen 114.

Within the ellipse-shaped lumen 306 are a first cavity 302 and second cavity 304 for a light transmission element or elements. The cavities 302 and 304 may extend longitudinally across the ellipse-shaped lumen 306 and defined by the interior wall of the ellipse-shaped lumen 306 and the arcs of the first lumen 112 and second lumen 114. The light transmission element or elements within the first cavity 302 and second cavity 304 may include, for example, fiber optic cables or bundles connected to the light post 140 proximally positioned on the body 102 of the hysteroscope 100. At the distal end of the barrel 150, the first cavity 302 and second cavity 304 may be sealed from fluid entry, for example, by a clear or transparent adhesive so that light may be transmitted from the light post 140 out the distal end of the barrel 150 and into the uterus 200. These light fibers may extend through the first cavity 302 and second cavity 304.

Exterior to the ellipse-shaped lumen 306 and within the interior wall of the outer tube or barrel 150, may be the third lumen 116 and fourth lumen 118. The third lumen 116 and fourth lumen 118 may be irrigation lumens, for example, positioned through the proximal body 102 and outside the ellipse-shaped lumen 306 within the distal outer tube or barrel 150 having open distal ends and proximal valves 220. The third lumen 116 and fourth lumen 118 may occupy areas created by the ellipse-shaped lumen 306 and its outer wall and the inner wall of the barrel 150. The irrigation lumens 116 and 118 are for the inflow of fluid and will be shown below to balance an inflow-to-outflow rate for the hysteroscope 100.

The third lumen 116 and the fourth lumen 118 for irrigation may be located laterally and symmetrically between the ellipse-shaped lumen 306 surrounding the first lumen 112 and the second lumen 116. The third lumen 116 and fourth lumen 118 may be defined, for example, as having a cross-section of a general reniform shape. Each of the third lumen 116 and fourth lumen 118 may define a concavely-shaped portion, or concavity, which contributes to the general reniform shape. The third lumen 116 and fourth lumen 118 may be constructed from stainless steel, or other similar materials.

Alternatively, the third lumen 116 and fourth lumen 118 may be defined by the area surrounding the barrel 150 and ellipse-shaped lumen 306 of the hysteroscope 100 and not by the general reniform shape. Additional inflow may be created by removing the general reniform shape. The cross-section for the third lumen 116 and fourth lumen 118 may be defined as a crescent-shape and substantially extend the length of the barrel 150.

A surgical tool 350 may be placed into the second lumen 114 for the working channel. The surgical tool 350 may be selected from a variety of different tools, for example, the surgical tool 350 may be a rotary morcellator, a reciprocating morcellator, or a morcellator having both reciprocal and rotary capabilities. When inserted, the surgical tool 350 may be maneuvered through the working channel and extend from the barrel 150 of the hysteroscope 100. The surgical tool 350 may fit through the entire length of the distal outer tube or barrel 150 of the hysteroscope 100. The tool 350 may include an outflow channel to receive fluid or other debris exiting the uterus 200. The diameter 352 of the tool 350 may be defined by the inner wall of the second lumen 114. An outer diameter 352 of the surgical tool 350 (for example, a morcellator) may be about 2.9 mm. Various configurations will be provided below.

FIG. 3B is a side view into a barrel 150 of the illustrative hysteroscope 100 without the surgical tool 350 according to one embodiment of the present disclosure. The geometric balance provided by the ellipse-shaped lumen 306 may unify the inflow volume of the third lumen 116 and fourth lumen 118 and the outflow provided by the second lumen 114 which provides for the outflow channel. The first lumen 112 may include the optic system 110, which may have a standardized diameter. The first cavity 302 and second cavity 304 may have the light element or elements. Typically, the outer diameter of the barrel 150 is 6.2 mm or smaller. In operating room situations, the outer diameter may range from 5 mm to 9 mm.

With reference to FIG. 3C, a side view into the barrel 150 of the illustrative hysteroscope 100 with the surgical tool 350 inserted according to one embodiment of the present disclosure is provided. The ellipse-shaped lumen 306 having the first lumen 112 may contain the optic system 110. The tool 350 may have an outer diameter that is an internal diameter of the second lumen 114, for example. Within the tool 350 may include an outflow channel for fluid or solid intake. Combining the first lumen 112 and second lumen 114 into the ellipse-shaped lumen 306 may provide structure stability when coupled to the internal diameter of the barrel 150. The first cavity 302 and second cavity 304 may have the light element or elements.

FIG. 3D is a side view into a cross-section of the barrel 150 with the surgical tool 350 inserted into the illustrative hysteroscope 100 according to one embodiment of the present disclosure. The taken cross-section lies close to the distal end of the barrel 150. The ellipse-shaped lumen 306 having the first lumen 112 may contain the optic system 110. Within the second lumen 114 housing the surgical tool 350 may be the outer diameter 352 of the surgical tool 350. An inner diameter 354 of the surgical tool 350 is shown and may define an outflow channel 356. This outflow channel 356 may receive liquids, tissue or the like from the uterus 200 while performing a surgical operation. The outflow channel 356 may also be part of the second lumen 114 without the tool 350 in diagnostic procedures. The first cavity 302 and second cavity 304 may have the light element or elements.

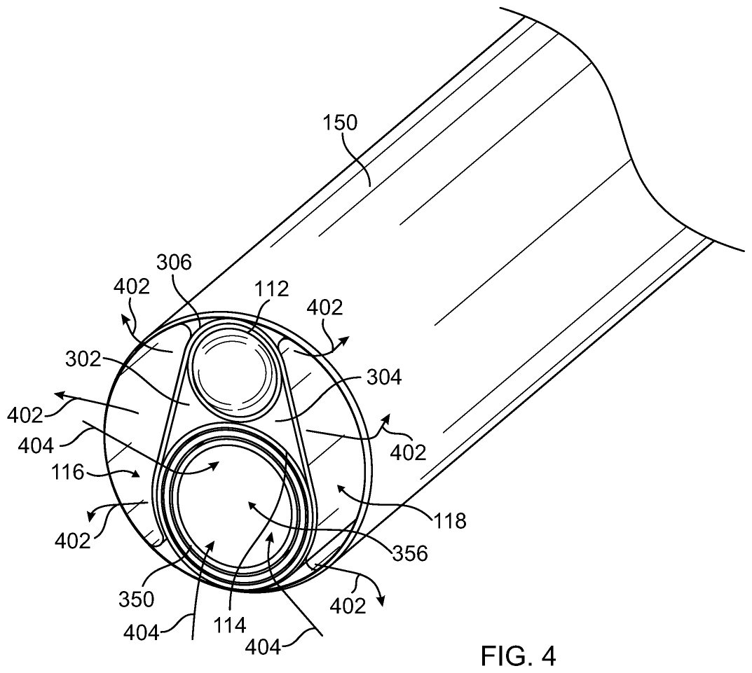

Turning to FIG. 4, a top perspective view of a cross-section of the barrel 150 of the illustrative hysteroscope 100 with the surgical tool 350 inserted showing a regulation of inflow 402 and outflow 404 of fluid according to one embodiment of the present disclosure is provided. Fluid coming into the uterus 200 from the irrigation channels of the third lumen 116 and fourth lumen 118 may be balanced with that of the outflow channel 356 of the surgical tool 350 within the second lumen 114. When the tool 350 is not within the second lumen 114, the outflow channel 356 of the hysteroscope 100 should maintain this balance as well.

In one embodiment, the third lumen 116 and fourth lumen 118 may be independent irrigation lumens or channels having openings at the distal end of the body 102 and valves 120 at their proximal portions to control the flow of fluid therethrough in order to keep the uterus 200 distended and pressure maintained with a fluid medium during diagnostic and therapeutic procedures. The inflow 402 may be provided through the third lumen 116 and fourth lumen 118 and flush the uterus 200 with fluid.

A fluid management system of the hysteroscope 100 may be used to regulate the inflow 402 and outflow 404. The system, for example, may include a pump that has a pressure regulator on it. Fluid management deficit may be tracked, that is, determining whether the patient is absorbing too much fluid. Operatively, the fluid management system pushes the fluid as inflow 402 into the channels of the third lumen 116 and fourth lumen 118 and out into the uterus 200. The outflow 404 may go through the second lumen 114 of the outflow channel 356 of the tool 350. A vacuum may be provided on the back side of the tool 350 to evacuate the fluid at the seal 270 of the proximate body 102. The balance, for example, would be to maintain from a control standpoint a large enough inflow 402 with an appropriate rate of outflow 404.

In one example, a hysteroscopy performed with fluid inflow 402 at a sufficient pressure, usually between 70 mm and 90 mm Hg of true intrauterine pressure, may bring about satisfactory uterus 200 distention. Depending on the amount of intraoperative bleeding, an adequate inflow 402 with separate channels of entry and egress may be used to have a clear operative field. As shown, the irrigation channels of the third lumen 116 and fourth lumen 118 may disperse fluids radially from the distal end of the barrel 150. Inflow 402 may also be directed over the first lumen 112 having the optical system 110 to maintain visibility as well as over the tool 350 when performing a procedure to flush tissue or other debris from the surgical site.

Balanced against these prerequisites may be a fluid inflow 402 overload and/or electrolyte imbalance as a consequence of intravasation of the fluid via the uterine vasculature. When arterial bleeding is encountered, a consequence of pressure relationships may occur. If arterial pressure exceeds that within the uterus 200, blood flow may hinder visualization through the optical system 110 of the first lumen 112. When pressure relationships are reversed, fluid may flow into the arterial tree, sometimes quite rapidly. Ideally, intracavitary pressure should equal mean arterial pressure.

Multiple configurations may exist with the ellipse-shaped lumen 306. Example data for inflow 402 and outflow 404 are shown within Table 1 below. These measurements should be considered for illustrative purposes and not limiting to the present disclosure.

TABLE 1

| | First | Outer | Inner | | | |
|---|---|---|---|---|---|---|
| Barrel | Channel | Diam- | Diam- | | | Inflow- |
| Outer | Optical | eter of | eter of | | | to- |
| Diameter | Diameter | Cutter | Cutter | Inflow | Outflow | Outflow |
| (mm) | (mm) | (mm) | (mm) | (mm\hat{ }2) | (mm\hat{ }2) | (mm\hat{ }2) |
| 7.80 | 1.80 | 5.01 | 4.57 | 14.30 | 16.40 | 0.87 |
| 8.00 | 1.80 | 5.21 | 4.77 | 14.85 | 17.87 | 0.83 |
| 6.60 | 1.80 | 3.81 | 3.37 | 10.98 | 8.92 | 1.23 |
| 6.80 | 1.80 | 4.01 | 3.57 | 11.53 | 10.01 | 1.15 |
| 7.00 | 1.80 | 4.21 | 3.77 | 12.08 | 11.16 | 1.08 |
| 7.20 | 1.80 | 4.41 | 3.97 | 12.64 | 12.38 | 1.02 |
| 5.80 | 1.80 | 3.01 | 2.57 | 8.78 | 5.18 | 1.69 |
| 6.00 | 1.80 | 3.21 | 2.77 | 9.33 | 6.03 | 1.55 |
| 6.20 | 1.80 | 3.41 | 2.97 | 9.88 | 6.93 | 1.43 |
| 6.40 | 1.80 | 3.61 | 3.17 | 10.43 | 7.89 | 1.32 |

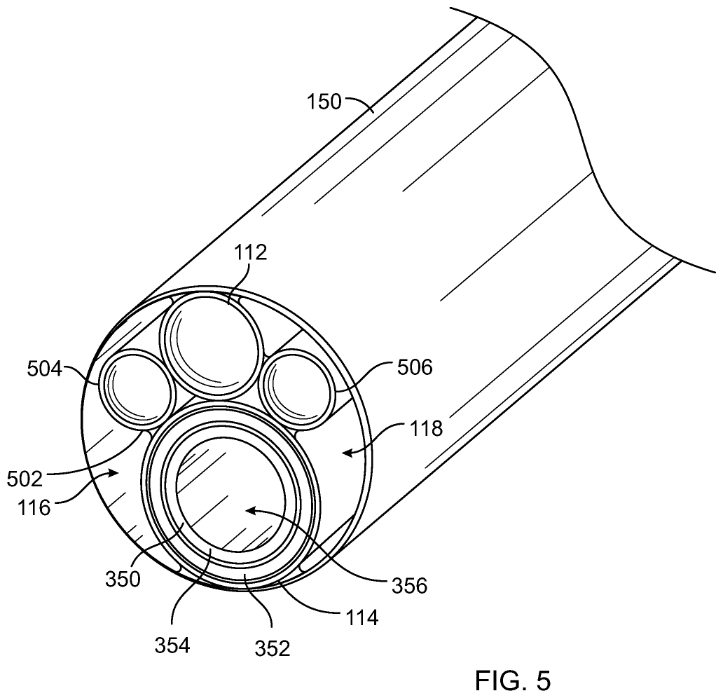

FIG. 5 is a top perspective view of a cross-section of a barrel 150 of another illustrative hysteroscope 100 having an amorphous-shaped lumen 502 according to one embodiment of the present disclosure. In the shown cross-section, the optical, luminescence, and working lumens or channels may be bound together creating an amorphous-shaped lumen 502 to provide a balance of inflow 402 and outflow 404 of fluids.

The amorphous-shaped lumen 502 may be permanently attached or coupled to the proximal body 102 of the hysteroscope 100, while the outer wall may be modular in a form of a sheath. In one embodiment, the amorphous-shaped lumen 502 may replace the cavities 302 and 304 that housed the lighting elements, with a first light lumen 504 and a second light lumen 506. The first light lumen 504 and the second light lumen 506 may take a generally cylindrical shape, for example, and extend longitudinally down the barrel 150.

The first light lumen 504 and the second light lumen 506 may include a light transmission element or elements, for example, by fiber optic cables or bundles connected to the light post 140 proximally positioned on the body 102 of the hysteroscope 100. At the distal end of the barrel 150, the first light lumen 504 and the second light lumen 506 may be sealed from fluid entry, for example, by a clear or transparent adhesive, so that light may be transmitted from the light post 140 out the distal end of the barrel 150 and into the uterus 200.

The amorphous-shaped lumen 502, as shown, may also incorporate the second lumen 114 housing the working channel. The inner diameter of the second lumen 114 may be the outer diameter 352 of the surgical tool 350. The tool 350 may be moved or rotated around the second lumen 114 which has the working channel. The inner diameter 354 of the tool 350 may define the outflow channel 356. This outflow channel 356 may receive outflow 404 from the uterus 200 while performing a diagnostic or surgical operation.

The amorphous-shaped lumen 502 may be formed from a combination of the first lumen 112 having the optical system 110, second lumen 114 for the working channel, and the first light lumen 504 and the second light lumen 506. The first lumen 112 may be superior to the first light lumen 504 and the second light lumen 506. The first light lumen 504 and the second light lumen 506 are symmetric around the first lumen 112 and second lumen 114. The lumens may extend longitudinally down the barrel 150 of the hysteroscope 100 towards the proximal body 102.

In the amorphous-shaped lumen 502, an arc of the first light lumen 504 may be coupled to arcs of the first lumen 112 and the second lumen 114. The second light lumen 506, similarly and symmetrically, may be coupled to arcs of the first lumen 112 and the second lumen 114 thus generating the amorphous-shaped lumen 502. The first lumen 112 may be connected to the second lumen 114 making the outer diameters of the first lumen 112 and second lumen 114 the internal diameter of the barrel 150 and the height of the amorphous-shaped lumen 502. The first lumen 112 may connect to an internal diameter of the barrel 150 with the second lumen 114 connecting to an opposite point across the barrel 150. These connections may extend the entire length of the barrel 150.

Outside the amorphous-shaped lumen 502, for example, may be the third lumen 116 and fourth lumen 118 which provide the irrigation channels. The third lumen 116 and fourth lumen 118 may be independent of one another with each having openings at their proximal end through their valves 120. These valves 120 may control the inflow 402 of fluid therethrough in order to keep the uterus distended and pressure maintained with a fluid medium during diagnostic and therapeutic procedures.

The third lumen 116 and fourth lumen 118 may symmetrically surround the amorphous-shaped lumen 502 extending past the first light lumen 504 and the second light lumen 506. In another configuration, the first light lumen 504 and the second light lumen 506 may extend and connect with the barrel 150 limiting the irrigation channels of the third lumen 116 and fourth lumen 118.

The inflow 402 and outflow 404 of the amorphous-shaped lumen 502 within the barrel 150 may be balanced through the fluid management system described above. The third lumen 116 and fourth lumen 118 may work with the outflow channel 356 either with the tool 350 or through the outflow channel 356 defined by the second lumen 114. The two independent irrigation or inflow channels associated with the valves 120 may work in conjunction with the outflow channel 356 to distend the uterus 200 during diagnostic (pathology identification) and therapeutic (pathology removal) procedures.

Multiple configurations may exist for the amorphous-shaped lumen 502. In one illustrative example, the barrel 150 may have an outer diameter of 5.80 mm and the first channel 112 having the rod lens may have a diameter of 1.80 mm. The second lumen 114 for the working channel having the tool 350 may have an inner diameter 354 of 2.06 mm and an outer diameter 352 of 3.18 mm. The inflow 402 from the third lumen 116 and fourth lumen 118 may be 6.64 mm\hat{ }2 and the outflow 404 from the outflow channel 356 may be 3.41 mm\hat{ }2. The inflow-to-outflow ratio may be 1.95 with the light fibers having 2.66 mm\hat{ }2.

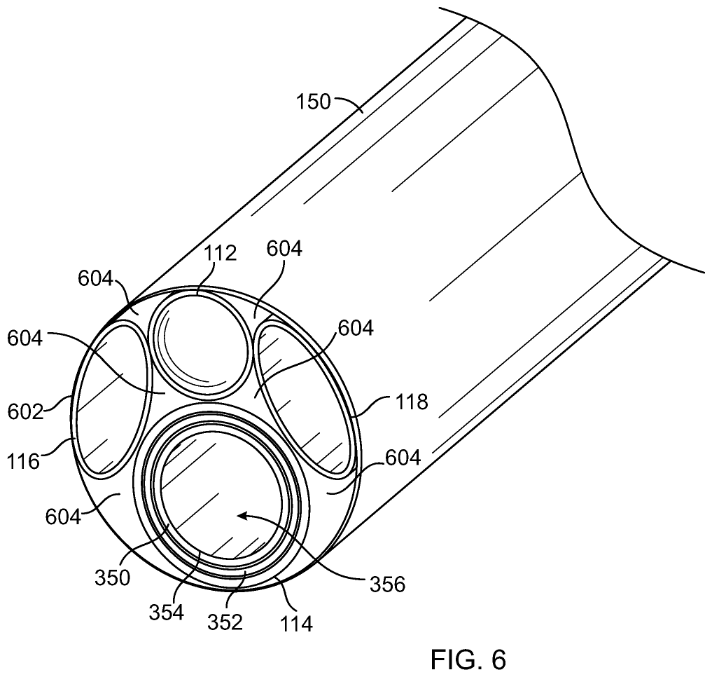

FIG. 6 is a top perspective view of a cross-section of a barrel 150 of another illustrative hysteroscope 100 having ellipse-shaped irrigation lumens 116 and 118 according to one embodiment of the present disclosure. The ellipse-shaped irrigation lumens 116 and 118 may provide a larger inflow 402 than circular shaped lumens. By providing ellipse-shaped irrigation lumens 116 and 118, the second lumen 114 may have an enlarged working channel for an outflow channel 356.

For surgical procedures, a tool 350 may be placed within the second lumen 114. The tool 350 may have an outer diameter 352 and an inner diameter 354. The inner diameter 354 may define the outflow channel 356. This outflow channel 356 may receive outflow from the uterus 200 while performing a surgical operation. Alternatively, the outflow channel 356 may be connected with the second lumen 114 absent the tool 350.

In the shown configuration, an amorphous-shaped lumen 602 may be created by the first lumen 112 and second lumen 114, which may be connected to one another. Their outer diameters combined may match the inner diameter of the barrel 150. At least a portion of their outer arcs may be coupled or connected to a top and bottom the inner diameter of the barrel 150. The first lumen 112 and second lumen 114 may be connected, for example, to one another substantially down the elongated barrel 150. The amorphous-shaped lumen 602 may give the barrel 150 a circular cross-section.

Symmetrically to the first lumen 112 and second lumen 114 may be the ellipse-shaped irrigation lumens 116 and 118. The lumens 116 and 118 may be connected with the sides of the first lumen 112 and second lumen 114 creating the amorphous-shaped lumen 602. The ellipse-shaped irrigation lumens 116 and 118 may extend to the inner diameter of the barrel 150, for example. The inflow 402 of the irrigation lumens 116 and 118 may be close to or match the outflow 404 of the working channel in the second lumen 114. By having the ellipse-shaped irrigation lumens 116 and 118, as shown, the size of the second lumen 114 may be greater than that of configurations having circular-shaped irrigation lumens. The irrigation lumens 116 and 118 may provide a distribution of inflow 402 that is uniform within the uterus 200 and above the outflow channel 356 such that debris or outflow 404 may be naturally channeled therethrough.

A number of cavities 604 may be formed from the amorphous-shaped lumen 602 and the outer barrel 150. These cavities 604 may be filled with a light transmission element or elements. The cavities 604 may substantially extend down the barrel 150 of the hysteroscope 100 and within the amorphous-shaped lumen 602. The light transmission element or elements within the cavities 604 may include, for example, fiber optic cables or bundles connected to the light post 140 proximally positioned on the body 102 of the hysteroscope 100. The placement of the light elements may allow a user to view areas around the first lumen 112 having the optical system 110 and the second lumen 114 for the working channel.

Multiple configurations may exist for the amorphous-shaped lumen 602. In one illustrative example, the barrel 150 may have an outer diameter of 5.80 mm and the first channel 112 having the rod lens may have a diameter of 1.80 mm. The second lumen 114 with the tool 350 and the working channel may have an inner diameter of 2.40. The inflow 402 from the third lumen 116 and fourth lumen 118 may be 5.42 mm\hat{ }2 and the outflow 404 may be 4.52 mm\hat{ }2. The inflow-to-outflow ratio may be 1.20.

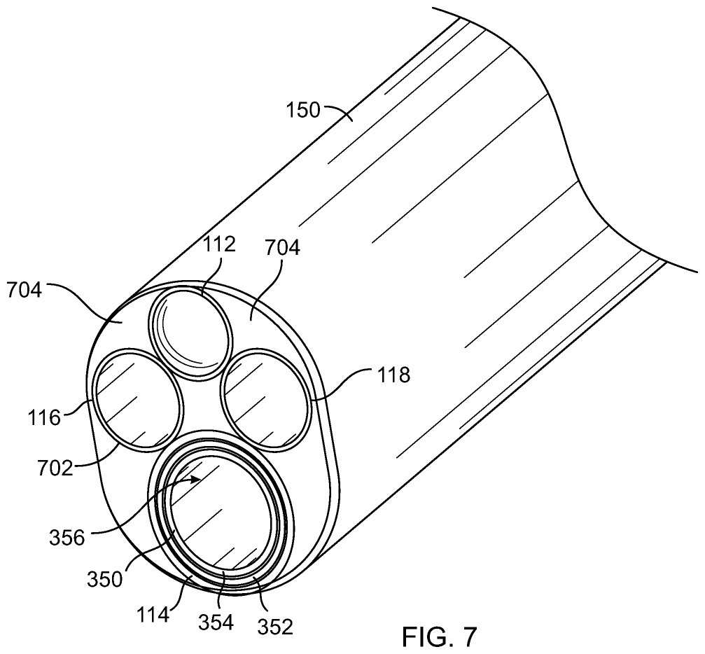

FIG. 7 is a top perspective view of a cross-section of another illustrative hysteroscope 100 having an oblong-shaped barrel 150 according to one embodiment of the present disclosure. An amorphous-shaped lumen 702 within the barrel 150 may be defined by the first lumen 112 housing the optical system 110 and second lumen 114 for the working channel with the third lumen 116 and fourth lumen 118 symmetrically distributed between them. The lumens may have, for example, circular cross-sections with varying diameters and the irrigation lumens 116 and 118 may have equal diameters that extend to the inner diameter of the barrel 150.

The outer barrel 150 may have an oblong-shaped cross-section that extends towards the body 102 of the hysteroscope 100. This shape may constrain the lumens therein keeping them proportional to one another. For example, the inflow 402 of the third lumen 116 and fourth lumen 118 should be similar or close to the outflow 404 of the second lumen 404 because of the geometric configuration of the barrel 150. The inflow 402 produced by the third lumen 116 and fourth lumen 118 may be distributed radially from each of the lumens such that a user of the hysteroscope 100 may have a clear view through the optical system 100.

A middle portion may be formed between the lumens and within the amorphous-shaped lumen 702. This along with the lower portions between the lumens 116 and 118, barrel 150, and second lumen 114 may be sealed or fluid tight. Cavities 704, which are formed between the barrel 150, first lumen 112, and the irrigation lumens 116 and 118 may provide a lighting element or elements and be connected with the optical system 110.

Multiple configurations may exist for the amorphous-shaped lumen 702. In one illustrative example, the first channel 112 having the rod lens may have a diameter of 1.80 mm. The oblong-shaped barrel 150 may have a width of 5.80 mm and a height of 7.00 mm. The second lumen 114 having the tool 350 with its outer diameter 352 and the outflow channel 356 may have an inner diameter 354 of 2.40 mm. The irrigation lumens 116 and 118 may both provide an inflow 402 and have a channel diameter of 2.01 mm. The inflow 402 from the third lumen 116 and fourth lumen 118 may be 6.36 mm\hat{ }2 and the outflow 404 may be 4.52 mm\hat{ }2. The inflow-to-outflow ratio may be 1.41.

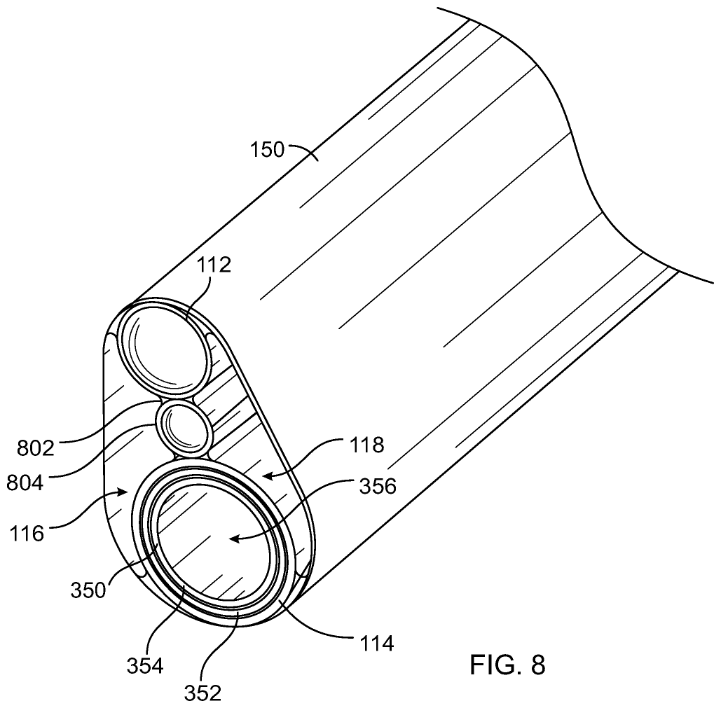

FIG. 8 is a top perspective view of a cross-section of another illustrative hysteroscope 100 having a pear-shaped barrel 150 according to one embodiment of the present disclosure. A compound-shaped lumen 802 within the barrel 150 may be made of a first lumen 112 which is connected to a luminescence lumen 804. The first lumen 112 may house the optical system 110. The luminescence lumen 804 may be connected to the second lumen 114 to form the compound-shaped lumen 802 which is centralized in the pear-shaped barrel 150.

By having the luminescence lumen 804 centrally within the compound-shaped lumen 802, light may be distributed radially from the distal end such that areas with the cavity may be viewed by the optical system 110. For example, this may allow a user of the hysteroscope 100 to view inflow 402 and outflow 404 of liquids. Light fibers within the luminescence lumen 804 may be positioned through the proximal body 102 and extend through the compound-shaped lumen 802 within the distal outer tube having a fluid-sealed distal end and a proximal light post 140 attached to a light source. The compound-shaped lumen 802 may be a three-tiered structure providing support for the barrel 150.

The second lumen 114 may have an inner diameter similar to the outer diameter 352 of the tool 350. The third lumen 116 and fourth lumen 118 having the inflow 402 may be symmetric to the compound-shaped lumen 802. Outflow 404 may be provided by the working channel within the second lumen 114 which may be defined by the inner diameter 354 of the tool 350. The geometric constraints of the pear-shaped barrel 150 and compound-shaped lumen 802 may regulate the inflow-to-outflow rates. As an example, a surface area created by the diameter of the outflow channel 356 may be similar to or match the area of the lumen 116 and fourth lumen 118.

The compound-shaped lumen 802 may be attached to the barrel 150 of the hysteroscope 100, while the outer wall may be modular in a form of a sheath. The diameters of the three lumens may connect from one end to the next to define the height of the compound-shaped lumen 802, and thus, the height or internal diameter of the pear-shaped barrel 150.

One advantage of the pear-shaped barrel 150 with the compound-shaped lumen 802 therein is that the pear-shaped barrel 150 may match the anatomy of the cervix 202. It will be understood that while the various lumens employed within the lumen of the barrel 150 have been described and shown in a particular configuration relative to one another, the various lumens may be arranged in alternative orientations depending on geometric constraints and still be within the scope of the present disclosure. While the various barrels 150 are shown as having cross-sectional shapes that are circular, oblong or pear, they may employ any regular or irregular cross-sectional shapes.

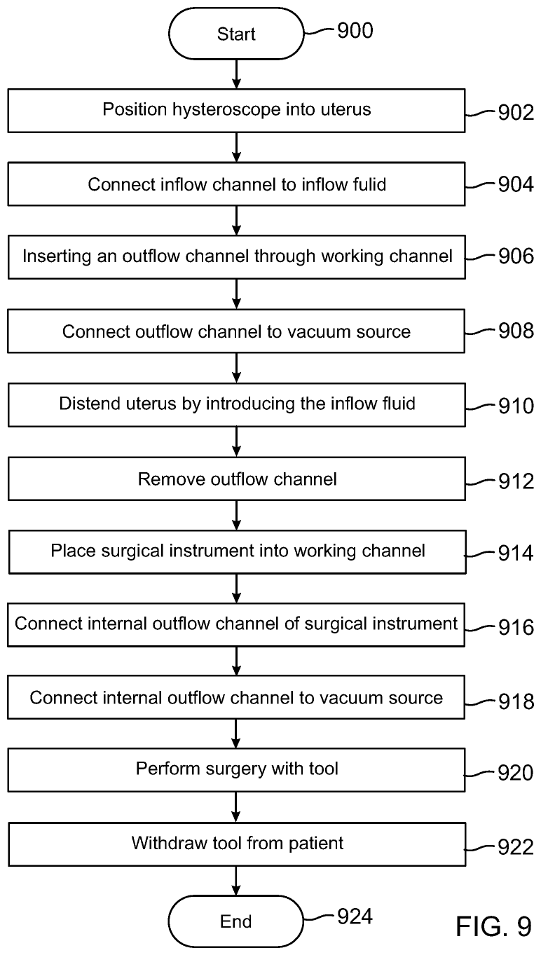

FIG. 9 is an exemplary flow chart showing illustrative processes for accessing an internal site in a patient's uterus 200 according to one embodiment of the present disclosure. It will be understood that various, or alternative, processes may be used depending on the patient's particular situation. For example, hysteroscopes 100 may include different tools 350 depending on the type of procedure. The processes may begin at block 900.

At block 902, the hysteroscope 100 may be positioned into the uterus 200. The distal portion of the insertion section 102 of the hysteroscope 100 having the barrel 150 may be inserted through the vagina and cervix 202 and into the uterus 200 of the patient. Dilation processes may be used accordingly.

The hysteroscope 100 may be connected with the inflow channel to inflow fluid at block 904. This may be through the third lumen 116 and fourth lumen 118 presented above. Both lumens may extend through and to a distal end of the insertion section 104 of the hysteroscope 100. As shown in the cross-sections above, the third lumen 116 and fourth lumen 118 were typically located laterally and symmetrically between the first lumen 112 which housed the optical system 110 and the second lumen 114 for the working channel. The lumens 116 and 118 may be independent irrigation lumens or channels having openings at the distal end of the body 102 and valves 120 at their proximal portions to control the flow of inflow 402 therethrough in order to keep the uterus distended and pressure maintained with a fluid medium during diagnostic and therapeutic procedures.

At block 906, an outflow channel 356 may be inserted into the hysteroscope 100 through the working channel. In certain embodiments, as shown in FIG. 3B, the system of the present disclosure included an outflow channel 356 sized for insertion through the working channel of the hysteroscope 100. At block 908, the outflow channel 356 may be connected to a vacuum source. This may be connected through the seal 170 at a proximal portion of the body 102 of the hysteroscope 100.

At block 910, the uterus 200 may be distend through inflow 402 provided by the third lumen 116 and a fourth lumen 118, or other irrigation channel described above. Fluid inflow 402, via lumens 116 and 118, and outflow 404, via the outflow channel 356 positioned within the second lumen 114 for the working channel, may be manipulated in order to distend the uterus 200. After achieving an optimum fluid balance, the interior of the uterus 200 and pathology may be visually investigated through the first lumen 112 having the optical system 110.

After the diagnostic procedure, the outflow channel 356 may be removed from the working channel within the second lumen 114 at block 912. At block 914, and if a procedure may be required, a surgical instrument or tool 350 may be placed into the working channel, as shown in FIG. 3C, and may be connected through the seal 170 at block 916.

At block 918, the internal outflow channel 356 of the surgical tool 350 may be connected to the vacuum source. The procedure may be used to balance the fluid inflow 402 and outflow 404. At block 920, a surgery may be performed with the tool 350. When the tool 350 is fully inserted into the body 102 of the hysteroscope 100, a set length of the distal portion of the tool 350, which may include cutting windows, may extend beyond the distal end of the hysteroscope 100 within the uterus 200. The tool 350 may then be activated by actuating a leaf spring trigger in the handle of the tool 350 and concurrently actuating the momentary switch of the tool 350 using a finger, for example, a finger of the same hand of the user grasping the handle and leaf spring trigger of the tool 350.

The user may then remove the pathology with the tool 350 while simultaneously visualizing the interior of the uterus 200 and pathology through the lumen 112 of the optical system 110. After removal of the pathology, at block 922, the hysteroscope 100 and tool 350 may be withdrawn from the patient. At block 924, the processes may end.

The foregoing description is provided to enable any person skilled in the relevant art to practice the various embodiments described herein. Various modifications to these embodiments will be readily apparent to those skilled in the relevant art and generic principles defined herein may be applied to other embodiments. Thus, the claims are not intended to be limited to the embodiments shown and described herein, but are to be accorded the full scope consistent with the language of the claims, wherein reference to an element in the singular is not intended to mean “one and only one” unless specifically stated, but rather “one or more.” All structural and functional equivalents to the elements of the various embodiments described throughout this disclosure that are known or later come to be known to those of ordinary skill in the relevant art are expressly incorporated herein by reference and intended to be encompassed by the claims. Moreover, nothing disclosed herein is intended to be dedicated to the public regardless of whether such disclosure is explicitly recited in the claims.

What is claimed is:

1. A hysteroscope comprising:
a proximal body from which a multi-lumened elongated outer tube extends;
an ellipse-shaped lumen positioned inside said outer tube;
an optical lumen disposed within said ellipse-shaped lumen;
a working lumen disposed within said ellipse-shaped lumen;
light fibers positioned within said ellipse-shaped lumen;
a first irrigation lumen positioned outside said ellipse-shaped lumen and within said outer tube; and
a second irrigation lumen positioned outside said ellipse-shaped lumen and within said outer tube.

2. The hysteroscope of claim 1, wherein a diameter of said optical lumen combined with a diameter of said working lumen extend to a top and bottom of said ellipse-shaped lumen.

3. The hysteroscope of claim 1, wherein said ellipse-shaped lumen is defined by an arc of said optical lumen and working lumen and tangent lines between them.

4. The hysteroscope of claim 1, wherein said optical lumen and said working lumen within said ellipse-shaped lumen form at least two cavities for said light fibers.

5. The hysteroscope of claim 1, wherein said working lumen is inferior to said optical lumen within said ellipse-shaped lumen.

6. The hysteroscope of claim 1, wherein said working lumen is connected to said optical lumen within said ellipse-shaped lumen.

7. The hysteroscope of claim 1, wherein said first irrigation lumen and second irrigation lumen extend from both sides of said ellipse-shaped lumen to said outer tube.

8. The hysteroscope of claim 1, wherein inflow of said first irrigation lumen and second irrigation lumen is balanced with outflow from said working lumen.

9. A hysteroscopy system comprising:
a body; and
a multi-lumened elongated outer tube extending from said body, said outer tube comprising:
an optical lumen extending through said outer tube;
a working lumen extending through said outer tube;
at least one luminescence extending through said outer tube;
a first irrigation lumen positioned within said outer tube;
a second irrigation lumen positioned said outer tube.

10. The hysteroscopy system of claim 9, comprising a first luminescence and second luminescence extending through said outer tube having a fluid-sealed distal end, wherein said optical lumen, working lumen, first luminescence and second luminescence are bound together into an amorphous-shaped lumen within said outer tube.

11. The hysteroscopy system of claim 10, wherein said amorphous-shaped lumen within said outer tube is surrounded by said first irrigation lumen and second irrigation lumen.

12. The hysteroscopy system of claim 9, wherein said first irrigation lumen and second irrigation lumen are ellipses-shaped with said at least one luminescence disposed between said lumens.

13. The hysteroscopy system of claim 9, wherein said multi-lumened elongated outer tube extending from said body to a distal portion is oblong-shaped.

14. The hysteroscopy system of claim 12, wherein a first luminescence is positioned between said oblong-shaped outer tube, first irrigation lumen and optical lumen and a second luminescence is positioned between said oblong-shaped outer tube, second irrigation lumen and optical lumen.

15. The hysteroscopy system of claim 9, wherein said multi-lumened elongated outer tube extending from said body to a distal portion is pear-shaped.

16. The hysteroscopy system of claim 15, comprising a luminescence extending through said outer tube having a fluid-sealed distal end, wherein said working lumen is inferior to said luminescence, said luminescence inferior to said optical lumen.

17. The hysteroscopy system of claim 16, wherein said working lumen, first luminescence and second luminescence are bound together into a compound-shaped lumen within said outer tube.

18. The hysteroscope of claim 16, comprising a fluid management system regulating inflow of said first irrigation lumen and second irrigation lumen with outflow from said working lumen.

19. A method of accessing an internal site in a patient's uterus comprising:
positioning said following lumens of a hysteroscope into a patient's uterus: an ellipse-shaped lumen having an optical lumen and working lumen positioned therein; and at least one irrigation lumen positioned outside said ellipse-shaped lumen;
introducing inflow fluid into said patient's uterus through said at least one irrigation lumen and thereby distending said patient's uterus; and
suctioning at least a portion of said inflow fluid out of said uterus through said working lumen.

20. The method of claim 19, comprising:
advancing a surgical instrument through said working lumen and into said patient's uterus; and
suctioning inflow fluid from said patient's uterus through an internal outflow channel of said surgical instrument.
